(19) United States
(12) Patent Application Publication
(10) Pub. No.: US 2023/0339837 A1
(43) Pub. Date: Oct. 26, 2023

(54) CONSTRUCTION METHOD AND APPLICATION OF MICROTUBE-BASED IONIC LIQUID COLLOID/WATER INTERFACE

(71) Applicant: East China Normal University, Shanghai (CN)

(72) Inventors: Limin ZHANG, Shanghai (CN); Yang TIAN, Shanghai (CN); Zehui CHEN, Shanghai (CN)

(21) Appl. No.: 18/135,893

(22) Filed: Apr. 18, 2023

(30) Foreign Application Priority Data
Apr. 20, 2022 (CN) 202210416331.8

(51) Int. Cl. C07C 51/41; C09K 23/00

(52) U.S. Cl. CPC C07C 51/412; C09K 23/00

(57) ABSTRACT

The present disclosure belongs to the technical field of liquid/liquid interface electrochemistry and analytical chemistry, and specifically provides construction of a microtube-based ionic liquid colloid/water interface and use. In the present disclosure, the construction method of an ionic liquid colloid/water interface with a high stability and a desirable selectivity includes the following steps: adding a poly(ionic liquid) into an ionic liquid to form an ionic liquid colloid, to enhance an interfacial stability of an organic phase; and adding a potassium ionophore into the organic phase to form a selective ionic liquid colloid/water interface. In this way, the ionic liquid colloid/water interface with a high stability and a desirable selectivity is constructed. The interface is applied to the detection of K⁺ in a cerebral cortex, and is of great significance for studying a behavior of the K⁺ in vivo and a relationship of the K⁺ with diseases.

### CROSS REFERENCE TO RELATED APPLICATION

This patent application claims the benefit and priority of Chinese Patent Application No. 202210416331.8, filed with the China National Intellectual Property Administration on Apr. 20, 2022, the disclosure of which is incorporated by reference herein in its entirety as part of the present application.

### TECHNICAL FIELD

The present disclosure belongs to the technical field of ion-selective sensing and detection, and in particular relates to construction method of a microtube-based ionic liquid colloid/water interface, and an electrochemical analysis method for detecting potassium ions by differential pulse voltammetry (DPV).

### BACKGROUND

Currently, as one of the most challenging major scientific issues, brain science researches help to understand the nature of complex physiological processes and the pathological mechanisms of brain diseases in the brain. The transmission of brain nerve signals is closely related to the participation of neurochemicals such as neurotransmitters, free radicals, and ions. An imbalance of these neurochemicals can lead to a variety of brain diseases including psychiatric disorders (such as depression) and neurodegenerative diseases (such as Alzheimer's disease). Therefore, it is of great significance for the prevention, diagnosis, and treatment of brain diseases by studying the molecular mechanisms of neurophysiology and neuropathology. Common brain science research methods include nuclear magnetic resonance (NMR) imaging, electrophysiology, and high-resolution fluorescence imaging. Electrochemical analysis has great advantages in the implantable in vivo detection of brain chemicals due to its high sensitivity, desirable spatial and temporal resolution, and easy miniaturization of electrodes. In the implantable in vivo detection, in vivo electrochemical analysis of liquid/solid interface based on voltammetry is widely used, such as micron-modified electrodes of carbon fibers, gold, platinum and the like. However, non-electroactive substances have large overpotentials and redox potentials that exceed the decomposition potential of water, making the detection of non-electroactive substances based on voltammetry in aqueous solutions still a huge challenge.

As a new type of interface, the liquid/liquid interface has great advantages in the detection of non-electroactive substances. The liquid/liquid interface relies on electrical signals generated by the migration of ions at the interface instead of electrical signals generated by a redox reaction. Traditional liquid/liquid interfaces have an organic phase, including supporting electrolytes and an organic solvent such as 1,2-dichloroethane and nitrobenzene. However, there is intracranial pressure in the rat brain, making the stability of the interface worse. To improve the stability of the liquid/liquid interface, polyvinyl chloride (PVC) is generally added to solidify the organic phase. This makes the conductivity of the organic phase worse, such that an ion transmission rate slows down, resulting in a decrease of detection sensitivity. Therefore, it remains a great challenge to conduct implantable detection of non-electroactive substances in the rat brain using a liquid/liquid interface.

### SUMMARY

Aiming at the above-mentioned problems in the prior art, the present disclosure provides an ionic liquid colloid/water interface. The interface has high stability, desirable selectivity, and strong ability to resist protein pollutions. In the present disclosure, in order to obtain the ionic liquid colloid/water interface with high stability, desirable selectivity, and strong ability to resist protein pollutions, the following technical means are specifically adopted. To enhance the stability of the interface, a poly(ionic liquid) is added into an ionic liquid for curing to form an ionic liquid colloid. A potassium ionophore is introduced into the ionic liquid colloid, to selectively assist the migration of potassium ions from an aqueous phase to an organic phase. In addition, proteins are hydrophilic and have a large molecular weight, and require high energy to migrate from the aqueous phase to the organic phase. Therefore, the liquid/liquid interface has excellent resistance to the protein pollutions. Eventually, the liquid/liquid interface is applied to the selective and accurate implantable detection of potassium ion levels in the rat brain.

The present disclosure provides a construction method of a microtube-based ionic liquid colloid/water interface, including the following steps:

(1) conducting synthesis of a hydrophobic ionic liquid with a wide interface potential window and screening;

(2) conducting synthesis of a poly(ionic liquid);

(3) conducting synthesis of a potassium ionophore;

(4) adding the poly(ionic liquid) obtained in step (2) and the potassium ionophore obtained in step (3) into the ionic liquid obtained in step (1) to obtain an ionic liquid colloid; and

(5) filling a microtube with the ionic liquid colloid obtained in step (4) to construct the ionic liquid colloid/water interface.

Further, in step (1), the ionic liquid is selected from the group consisting of C₂M, C₄M, and C₁₀M, which have structures shown in the following formula (a):

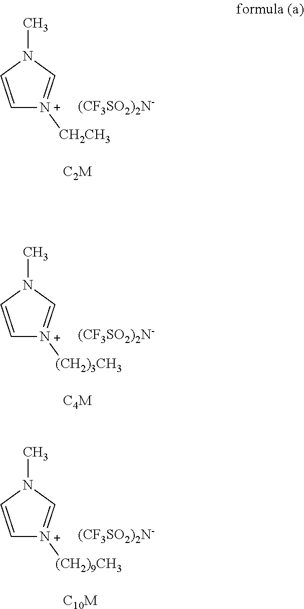

Further, in step (2), the poly(ionic liquid) is poly(1-butyl-3-vinylimidazolium bis(trifluoromethanesulfonyl)imide, and has a structure shown in formula (b):

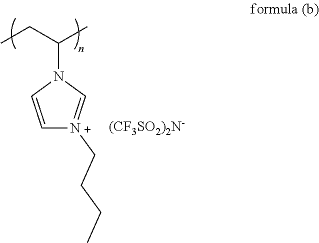

Further, the poly(1-butyl-3-vinylimidazolium bis(trifluoromethanesulfonyl)imide is obtained by conducting self-polymerization on a 1-butyl-3-vinylimidazolium bromide monomer and anion exchange with lithium bis(trifluoromethane)sulfonimide (LiTFSI).

Further, in step (3), the potassium ionophore has structure shown in formula (c):

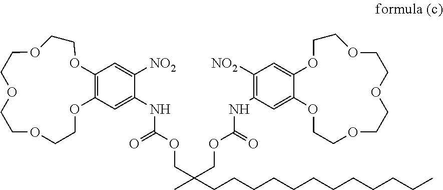 [SMILES: CCCCCCCCCCCCC(C)(COC(=O)Nc1cc2c(cc1[N+](=O)[O-])OCCOCCOCCOCCO2)COC(=O)Nc1cc2c(cc1[N+](=O)[O-])OCCOCCOCCOCCO2]

Further, in step (4), a preparation method of the ionic liquid colloid includes: mixing the poly(1-butyl-3-vinylimidazolium bis(trifluoromethanesulfonyl)imide with the ionic liquid to conduct solidification at a ratio of (0.5-3):1 (preferably 1:1), and adding the potassium ionophore, such that the potassium ionophore has a concentration of 30 mM, to obtain the ionic liquid colloid.

Further, in step (5), the laser-drawn microtube is filled with the ionic liquid colloid using a syringe.

Further, in step (6), a construction method of the ionic liquid colloid/water interface includes: placing the microtube containing the ionic liquid colloid into an aqueous solution to construct the ionic liquid colloid/water interface.

The present disclosure further provides an ionic liquid colloid prepared by the method.

The present disclosure further provides an ionic liquid colloid/water interface prepared by the method.

The present disclosure further provides use of the ionic liquid colloid/water interface in detection of K⁺ in vitro.

The present disclosure further provides a method for detecting a K⁺ concentration of a rat cerebral cortex using the ionic liquid colloid/water interface.

In the present disclosure, the potassium ionophore can bind K⁺ with high selectivity. Meanwhile, under a synergistic effect of an electric field, the K⁺ migrates from an aqueous phase to an organic phase, thereby obtaining a corresponding DPV curve and its linear range. In this way, changes in the K⁺ concentration can be detected based on DPV.

The present disclosure further provides a method for detecting K⁺ in vitro using a microtube-based ionic liquid colloid/water interface, including the following steps: adding a solution containing a certain concentration of K⁺ into an aqueous phase of the ionic liquid colloid/water interface, placing two silver wires coated with silver chloride in an ionic liquid colloidal phase and the aqueous phase, respectively; applying a voltage, such that the K⁺ migrates from the aqueous phase to an organic phase under a synergistic effect of an electric field and an ionophore; recording a current magnitude of the K⁺ migrating at the interface by differential pulse voltammetry (DPV), thereby achieving quantitative detection of the K⁺ in vitro.

The present disclosure further provides a method for detecting a K⁺ concentration in vitro through the ionic liquid colloid/water interface, including the following steps:

(1) placing the microtube filled with the ionic liquid colloid in an aqueous solution;

(2) placing two silver wires coated with silver chloride into the ionic liquid colloid and the aqueous solution, respectively;

(3) adding solutions containing different K⁺ concentrations to the aqueous solution; and

(4) conducting detection by DPV, and determining the K⁺ concentration by a peak current of an obtained DPV curve.

The method is suitable for detecting the K⁺ concentration in a linear range of 0.8 mM to 60 mM.

The method is suitable for detecting the K⁺ concentration with a minimum detection limit of 20 μM.

In a specific example of the present disclosure, the method for detecting a K⁺ concentration in vitro through the ionic liquid colloid/water interface includes:

(1) Plotting of a standard curve:

Solutions of different K⁺ concentrations are added to an aqueous phase that constitutes the ionic liquid colloid/water interface, such that K⁺ concentrations in the aqueous phase are 0.8 mM, 1 mM, 2 mM, 5 mM, 10 mM, 15 mM, 20.0 mM, 25 mM, 30 mM, 40 mM, 50 mM, and 60 mM. DPV curves at different concentrations are recorded, and relationship curves between a peak current and the K⁺ concentration of each group are plotted, to obtain a linear range of 0.8 mM to 60 mM.

(2) Determination of the K⁺ concentration in a sample solution

The microtube filled with the ionic liquid colloid is placed in a sample solution to form the ionic liquid colloid/water interface. The DPV curve is determined, and the K⁺ concentration in the sample solution is calculated according to a relationship between the K⁺ concentration and the peak current.

In the present disclosure, the DPV curve and its linear range obtained according to the above method can be used to detect changes of the K⁺ concentration in vitro.

The present disclosure further provides a method for detecting a K⁺ concentration of a rat cerebral cortex using a microtube-based ionic liquid colloid/water interface, including the following steps: implanting the microtube filled with the ionic liquid colloid into a rat cerebral cortex using a stereotaxic instrument, and forming an ionic liquid colloid/water interface in the rat brain; placing a silver wire coated with silver chloride and a reference electrode into an ionic liquid colloid phase and a rat cerebral dura mater, respectively; applying a voltage, such that K⁺ migrates from an aqueous phase to an organic phase under a synergistic effect of an electric field and an ionophore; determining a current magnitude of the K⁺ in the rat cerebral cortex and migrating at the interface by DPV, thereby achieving quantitative detection of the K⁺ in vivo.

The present disclosure further provides a method for detecting a K⁺ concentration of a rat cerebral cortex through the ionic liquid colloid/water interface, including the following steps:

(1) implanting the microtube containing the ionic liquid colloid into a rat cerebral cortex;

(2) placing an Ag/AgCl reference electrode into a cerebral dura mater; and

(3) conducting detection by DPV, and determining the K⁺ concentration in the cortex by a peak current of an obtained DPV curve.

The experimental rats are male Wistar rats weighing 200 g to 250 g.

The implanted cortex is L2/3 regions of a motor cortex, a visual cortex, and a sensory cortex separately.

In a specific example of the present disclosure, the method for detecting a K⁺ concentration of a rat cerebral cortex through the ionic liquid colloid/water interface includes:

(1) implanting the microtube filled with the ionic liquid colloid into a rat cerebral cortex using a stereotaxic instrument, forming an ionic liquid colloid/water interface in the rat brain, and recording a DPV curve of K⁺ migrating on the interface; and

(2) according to a peak current of the DPV curve and a standard curve, obtaining the K⁺ concentration in the cortex.

In the present disclosure, the microtube containing the ionic liquid colloid is separately implanted into the sensory cortex, motor cortex and visual cortex of the rat brain to form the ionic liquid colloid/water interface, and then DPV detection is conducted. The K⁺ content in different cortex regions of the rat brain is determined by the peak current of the DPV curve.

The present disclosure further provides a method for studying an anti-protein pollution ability of the ionic liquid colloid/water interface serving as an electrode.

In a specific example of the present disclosure, the method for studying an anti-protein pollution ability of the ionic liquid colloid/water interface includes:

a FITC-BSA solution with a concentration of 5 mg mL⁻¹ is added to the aqueous phase of the ionic liquid colloid/water interface, the DPV curve of K⁺ migrating at the interface is recorded at regular intervals, and a degree of pollution by fluorescent proteins at the interface is observed by a confocal microscope.

In the present disclosure, DPV curves and fluorescence imaging images obtained by the above method show that the ionic liquid colloid/water interface has a desirable ability to resist protein pollutions, and can be implanted in the rat brain for a long time without the interface being polluted by proteins.

The present disclosure further provides a method for studying a stability of the ionic liquid colloid/water interface serving as an electrode.

In a specific example of the present disclosure, the method for studying a stability of the ionic liquid colloid/water interface includes:

(1) observing a change of a liquid level before and after the microtube containing the ionic liquid colloid is implanted into the rat brain; and

(2) adding fluorescein into the ionic liquid colloid, and observing fluorescence imaging of a brain slice after the ionic liquid colloid/water interface is formed in the rat brain.

In the present disclosure, liquid level changes in the microtube and brain slice images obtained by the above method show that the ionic liquid colloid/water interface has desirable stability, and an intracranial pressure in the rat brain has little influence on the interface stability.

The present disclosure has the beneficial effects as follows: the selective ionic liquid colloid/water interface is constructed by adding potassium ionophore into the organic phase, and has a linear range of 0.8 mM to 60 mM and a minimum detection limit of 20 μM (S/N=3). The ionic liquid colloid/water interface has excellent anti-protein pollution ability, and still shows more than 83% current response to potassium ions after soaking in a BSA solution for 60 d. In addition, the interface acts as an electrode to selectively detect K⁺. Neurotransmitters, amino acids, metal ions, and other bioactive substances interfere little (<10%) with the K⁺ detection. Moreover, the interface has high detection stability after being implanted into the rat brain. Therefore, the ionic liquid colloid/water interface with high selectivity, desirable antifouling ability, and excellent stability can satisfy the detection of K⁺ concentration in vitro and in vivo.

### BRIEF DESCRIPTION OF THE DRAWINGS

FIG. 1 shows interface potential windows of ionic liquids with different hydrophobicities; where a scan rate is 10 mV/s;

FIGS. 2A-D show: (FIG. 2A) a schematic diagram of K⁺ migrating at a liquid/liquid interface; (FIG. 2B) DPV curves of different potassium ion concentrations in an aqueous solution when there is no potassium ionophore at the interface; (FIG. 2C) DPV curves of different potassium ion concentrations in an aqueous solution when there is a potassium ionophore at the interface, where a black dotted line represents a background current; and (FIG. 2D) a calibration curve of potassium ions detected by a ionic liquid colloid electrode;

FIGS. 3A-D show selectivity of the ionic liquid colloid electrode to metal ions, amino acids, anions and other biomolecules;

FIGS. 4A-C show: (FIG. 4A) fluorescent images of the ionic liquid colloid electrode placed in a 5 mg·mL⁻¹ FITC-BSA solution for 0 d, 5 d, 10 d, 25 d, 50 d, and 60 d; (FIG. 4B) DPV curves of the ionic liquid colloid electrode placed in a 5 mg·mL⁻¹ BSA solution during 0 d to 60 d; and (FIG. 4C) peak current ratios of DPVs before and after the ionic liquid colloid electrode is immersed in the BSA solution;

FIG. 5 shows slices of brain injury; and

FIG. 6 shows potassium ion concentrations in the motor cortex, the sensory cortex, and the visual cortex.

### DETAILED DESCRIPTION OF THE EMBODIMENTS

The present disclosure will be further described in detail with reference to the following specific examples and accompanying drawings. The process, conditions, and experimental methods for implementing the invention disclosure, excluding the content specially mentioned below, are known in the art. The present disclosure imposes no special limitation on the content.

### Example 1: Synthesis of Poly(1-butyl-3-vinylimidazolium Bis(trifluoromethanesulfonyl)imide

1-butyl-3-vinylimidazolium bromide (2.31 g, 10.00 mmol), 2,2-azobis(2-methylpropionitrile) (0.016 g, 0.10 mmol), and ethanol (5 mL) were stirred for 24 h in a 25 mL round bottom flask. The ethanol was removed by rotary evaporation, and excessive acetone was added to an obtained remaining mixture to generate a white precipitate, which was subjected to suction filtration. The precipitate was dried, dissolved in water, and 5 mL of an LiTFSI (3.00 g) solution was added dropwise until a sediment was completely sedimented, and then subjected to suction filtration, washing, and drying.

### Example 2: Determination of Potential Windows of Different Ionic Liquids

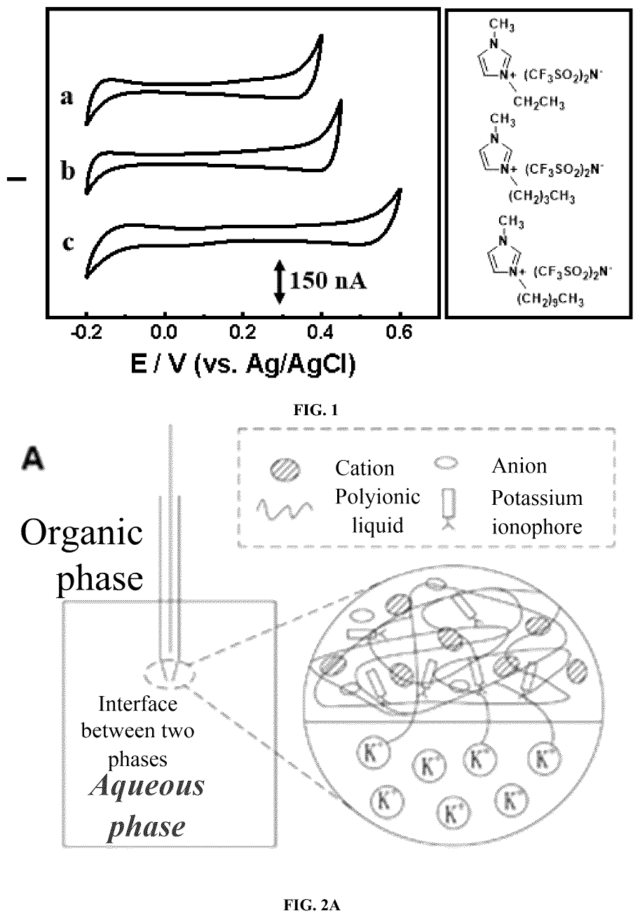

The ionic liquids C₂M, C₄M, and C₁₀M were separately placed in a hydrophobically-treated glass microtube, each obtained ionic liquid electrode was placed in a LiCl solution, and the potential windows of different ionic liquid/water interfaces were measured by cyclic voltammetry (CV). It was seen from FIG. 1 that the C₁₀M had the widest potential window up to 0.8 V, meeting the needs of subsequent ion detection. Therefore, the C₁₀M was used as an organic phase.

### Example 3: Ionic Liquid Colloid/Water Interface in Detection of K⁺ In Vitro

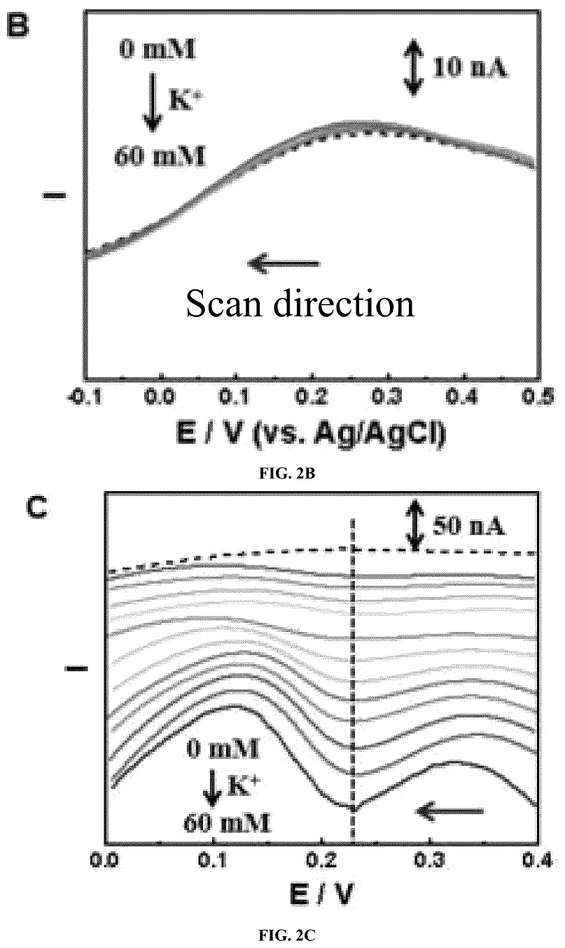

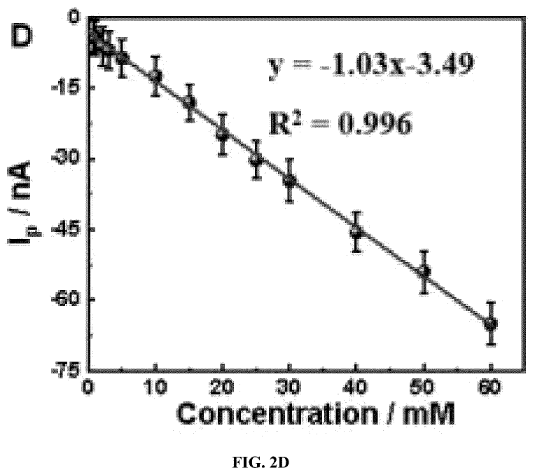

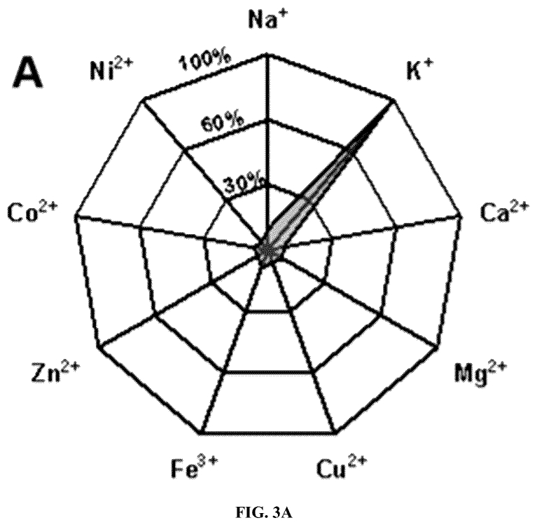

Solutions containing different K⁺ concentrations were added to an aqueous phase, and DPV curves of different K⁺ concentrations detected on the ionic liquid colloid/water interface without potassium ionophore were recorded. It was seen from FIG. 2B that when the interface was not assisted by potassium ionophores, K⁺ did not migrate from the aqueous phase to an organic phase, thus no migration current was generated. Solutions containing different K⁺ concentrations were added to an aqueous phase, and DPV curves of different K⁺ concentrations detected on the ionic liquid colloid/water interface with a potassium ionophore were recorded. A relationship curve between peak current and K⁺ concentration was plotted, and a linear range was determined to be 0.8 mM to 60 mM (FIG. 2C to FIG. 2D), which met the detection requirements of subsequent biological applications.

### Example 4: Selectivity Experiment

Selectivity experiments were conducted on the ionic liquid colloid/water interface in the presence of metal ions, anions, amino acids, and biologically-active substances with different concentrations.

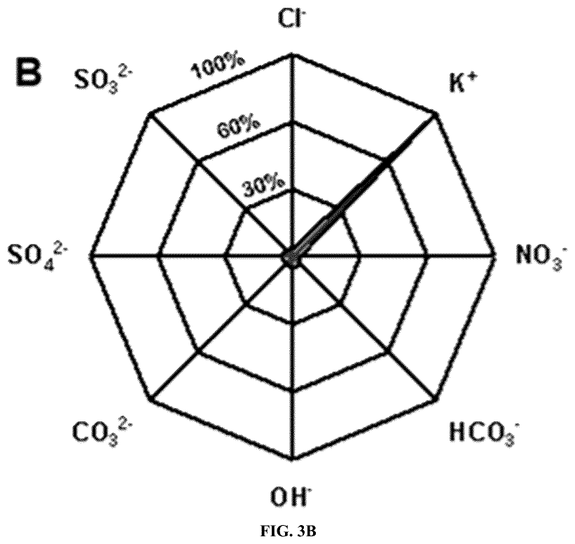

Metal ion neurotransmitters (10 mM of Na⁺, 1 mM of Ca²⁺ and Mg²⁺, and 10 μM of Cu²⁺, Fe³⁺, Zn²⁺, Co²⁺, Ni²⁺) (FIG. 3A), anions (10 μM of NO₃⁻, HCO₃⁻, OH⁻, CO₃²⁻, SO₄²⁻, SO₃²⁻, Cl⁻) (FIG. 3B), amino acids (10 μM of Phe, Met, Gly, Glu, Cys, Arg, Lys, Leu, Ser, Thr, Val) (FIG. 3C), and other bioactive substances (10 mM of glucose, and 10 μM of AA, DA, UA, 5-HT, DOPAC, lact) (FIG. 3D) were added to the aqueous solution. Corresponding peak currents were measured by DPV. 5 mM of K⁺ was added to each solution to obtain new corresponding peak currents. An I₍ᵢ₎/I_{K+} diagram was plotted according to each group of curves.

### Example 5: Anti-protein Pollution of the Interface

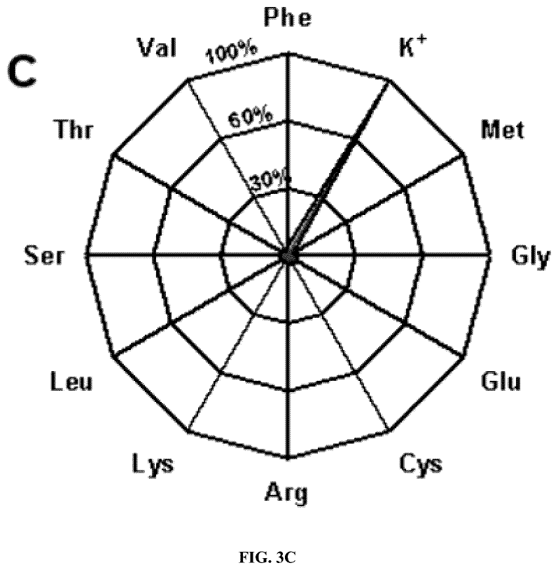

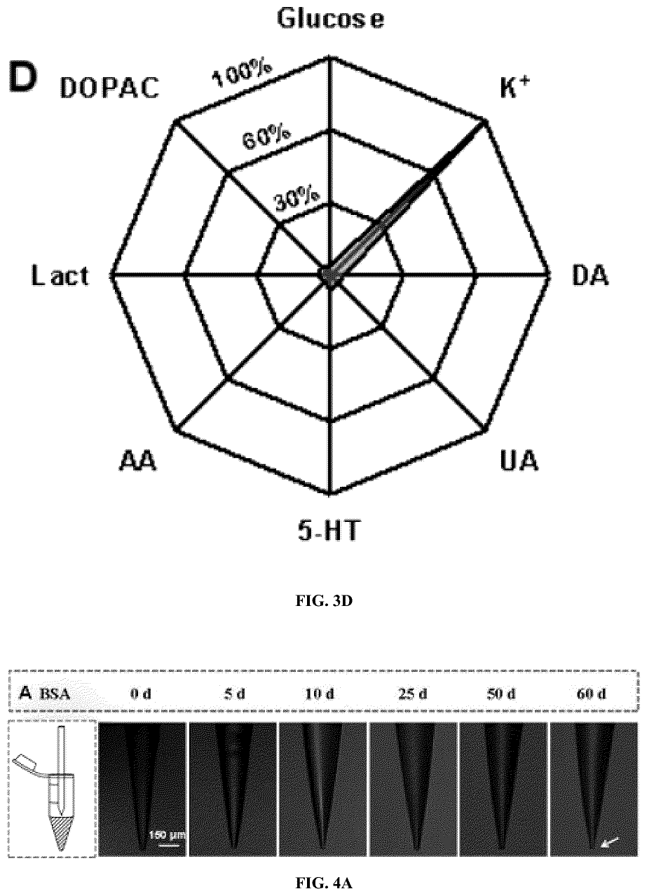

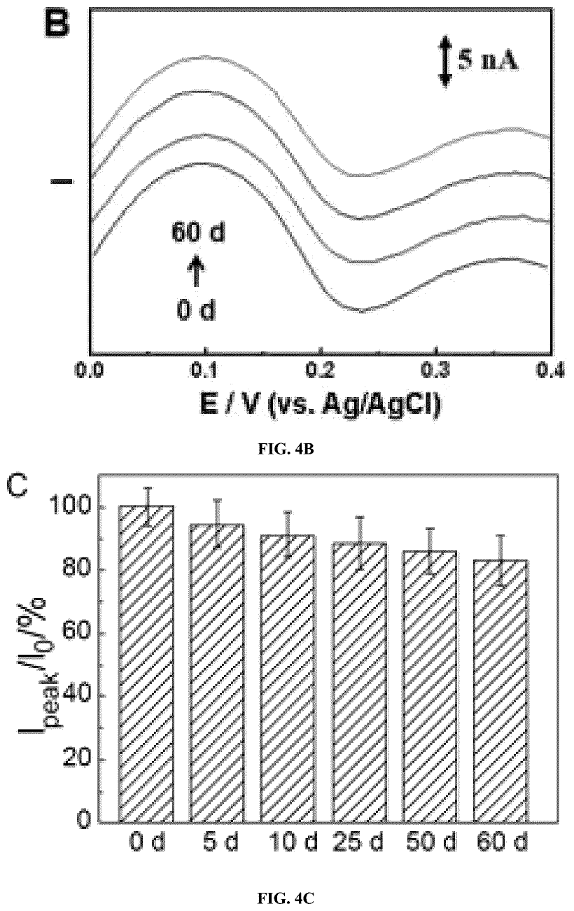

The microtube containing the ionic liquid colloid was inserted into an aqueous solution rich in 5 mg·mL⁻¹ of FITC-BSA to form an ionic liquid colloid/water interface. The protein pollution of the ionic liquid colloid/water interface was observed after the presence of FITC-BSA for 0 d, 5 d, 10 d, 25 d, 50 d, and 60 d. On day 60, the ionic liquid colloid/water interface began to be polluted to some extent (FIG. 4A). Meanwhile, DPV was conducted to measure the peak currents of K⁺ migrating at the ionic liquid colloid/water interface after the FITC-BSA existed for 0 d to 60 d. It was seen from FIG. 4B to FIG. 4C that even if the interface existed in the BSA solution for 60 d, the peak current still maintained at not less than 83% of an original value. The above data showed that the ionic liquid colloid/water interface had excellent resistance to protein pollutions.

### Example 6: Slices of Brain Injury

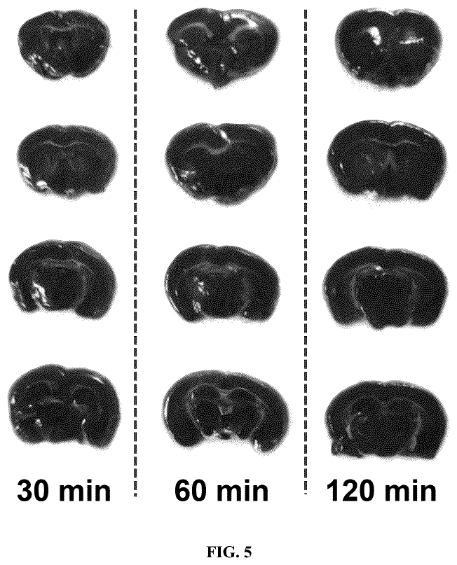

The microtube filled with the ionic liquid colloid was implanted into the rat brain, and the microtube was separately taken out after 30 min, 60 min, and 120 min, and the brain slices were made from the rat brain. The brain slices were soaked in a TTC solution and stained for 5 min to 10 min, and the injury of the brain slices was observed. As shown in FIG. 5, during the period of 30 min to 120 min after the microtubes were implanted into the rat brain, there was no obvious injury to the rat cerebral cortex.

### Example 7: Detection of K⁺ Content in Rat Cerebral Cortex

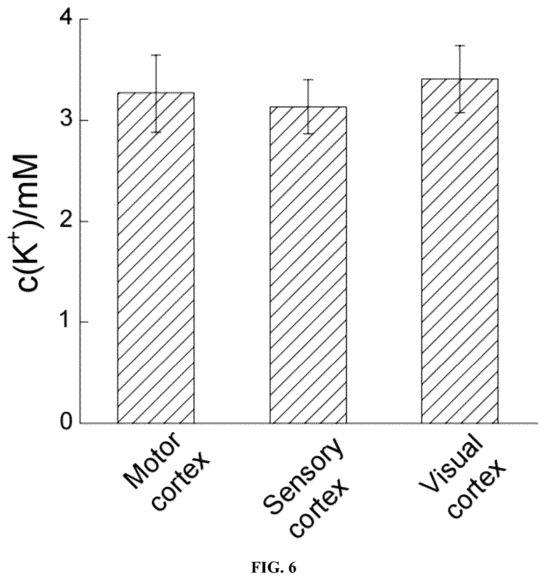

The microtube filled with the ionic liquid colloid was separately implanted into a motor cortex, a sensory cortex, and a visual cortex of the rat brain to construct the ionic liquid colloid/water interface in the rat brain, and potassium ion concentrations of different cortical layers were measured by DPV. According to a peak current of the DPV curve, the K⁺ concentration was calculated in the cortex. The motor cortex, the sensory cortex, and the visual cortex had potassium ion concentrations of 3.3 mM±0.37 mM, 3.1 mM±0.25 mM, 3.4 mM±0.31 mM, respectively (FIG. 6).

Protection content of the present disclosure is not limited to the described embodiments. Changes and advantages that can be easily figured out by persons skilled in the art without departing the spirit and scope of the present disclosure are included in the present disclosure and subject to the protection scope of the claims.

What is claimed is:

1. A construction method of a microtube-based ionic liquid colloid/water interface, comprising the following steps:
(1) conducting synthesis of an ionic liquid and screening;
(2) conducting synthesis of a poly(ionic liquid);
(3) conducting synthesis of a potassium ionophore;
(4) adding the poly(ionic liquid) obtained in step (2) and the potassium ionophore obtained in step (3) into the ionic liquid obtained in step (1) to prepare an ionic liquid colloid; and
(5) based on step (4), constructing an ionic liquid colloid/water interface.

2. The method according to claim 1, wherein in step (1), the ionic liquid has a structure shown in formula (a):

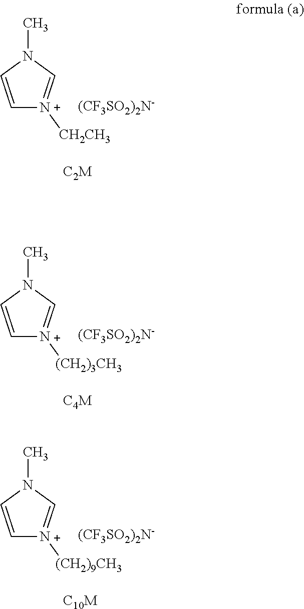

3. The method according to claim 1, wherein in step (2), the poly(ionic liquid) is poly(1-butyl-3-vinylimidazolium bis(trifluoromethanesulfonyl)imide, and has a structure shown in formula (b):

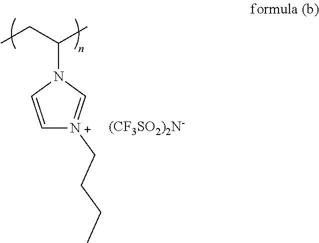

4. The method according to claim 3, wherein the poly(1-butyl-3-vinylimidazolium bis(trifluoromethanesulfonyl)imide is obtained by conducting self-polymerization on a 1-butyl-3-vinylimidazolium bromide monomer and anion exchange with lithium bis(trifluoromethane)sulfonimide (LiTFSI).

5. The method according to claim 1, wherein in step (3), the potassium ionophore has a structure shown in formula (c):

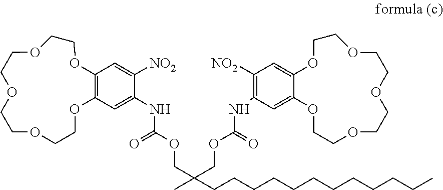 [SMILES: CCCCCCCCCCCCC(C)(COC(=O)Nc1cc2c(cc1[N+](=O)[O-])OCCOCCOCCOCCO2)COC(=O)Nc1cc2c(cc1[N+](=O)[O-])OCCOCCOCCOCCO2]

6. The method according to claim 1, wherein in step (4), a preparation method of the ionic liquid colloid comprises: mixing the poly(1-butyl-3-vinylimidazolium bis(trifluoromethanesulfonyl)imide with the ionic liquid to conduct solidification at a ratio of (0.5-3):1, and adding the potassium ionophore to obtain the ionic liquid colloid.

7. The method according to claim 1, wherein in step (5), a construction method of the ionic liquid colloid/water interface comprises: filling a microtube with the ionic liquid colloid using a syringe, and placing the microtube in an aqueous solution to construct the ionic liquid colloid/water interface.

8. An ionic liquid colloid/water interface constructed by the method according to claim 1.

9. The ionic liquid colloid/water interface according to claim 8, wherein in step (1), the ionic liquid has a structure shown in formula (a):

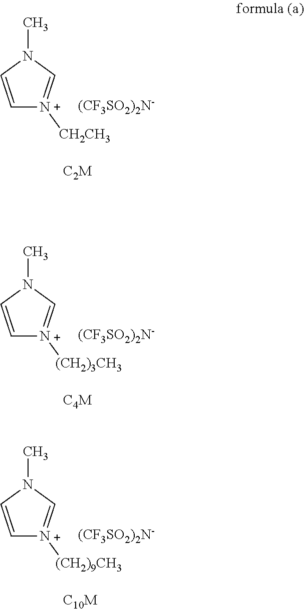

10. The ionic liquid colloid/water interface according to claim 8, wherein in step (2), the poly(ionic liquid) is poly(1-butyl-3-vinylimidazolium bis(trifluoromethanesulfonyl)imide, and has a structure shown in formula (b):

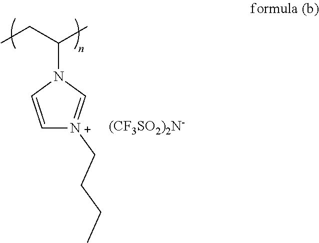

11. The ionic liquid colloid/water interface according to claim 10, wherein the poly(1-butyl-3-vinylimidazolium bis(trifluoromethanesulfonyl)imide is obtained by conducting self-polymerization on a 1-butyl-3-vinylimidazolium bromide monomer and anion exchange with lithium bis(trifluoromethane)sulfonimide (LiTFSI).

12. The ionic liquid colloid/water interface according to claim 8, wherein in step (3), the potassium ionophore has a structure shown in formula (c):

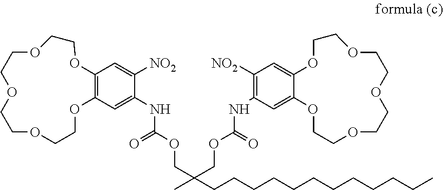 [SMILES: CCCCCCCCCCCCC(C)(COC(=O)Nc1cc2c(cc1[N+](=O)[O-])OCCOCCOCCOCCO2)COC(=O)Nc1cc2c(cc1[N+](=O)[O-])OCCOCCOCCOCCO2]

13. The ionic liquid colloid/water interface according to claim 8, wherein in step (4), a preparation method of the ionic liquid colloid comprises: mixing the poly(1-butyl-3-vinylimidazolium bis(trifluoromethanesulfonyl)imide with the ionic liquid to conduct solidification at a ratio of (0.5-3):1, and adding the potassium ionophore to obtain the ionic liquid colloid.

14. The ionic liquid colloid/water interface according to claim 8, wherein in step (5), a construction method of the ionic liquid colloid/water interface comprises: filling a microtube with the ionic liquid colloid using a syringe, and placing the microtube in an aqueous solution to construct the ionic liquid colloid/water interface.

15. A method for detecting K⁺ in vitro using a microtube-based ionic liquid colloid/water interface, comprising the following steps: adding a solution containing a certain concentration of K⁺ into an aqueous phase of the ionic liquid colloid/water interface according to claim 8, placing two silver wires coated with silver chloride in an ionic liquid colloidal phase and the aqueous phase, respectively; applying a voltage, such that the K⁺ migrates from the aqueous phase to an organic phase under a synergistic effect of an electric field and an ionophore; recording a current magnitude of the K⁺ migrating at the interface by differential pulse voltammetry (DPV), thereby achieving quantitative detection of the K⁺ in vitro.
